(19) United States
(12) Patent Application Publication
(10) Pub. No.: US 2004/0029170 A1
(43) Pub. Date: Feb. 12, 2004

(54) Method and device for the determination of analyte concentrations

(76) Inventors: Evelyn Wolfram, Julich (DE); Matthias Arnold, Weilerswist (DE); Andreas Franz, Wesseling (DE); Dirk Weuster-Botz, Dachau (DE); Christian Wandrey, Julich (DE)

Correspondence Address: Joseph W Berenato, Liniak Berenato & White, Suite240, 6550 Rock Spring Drive, Bethesda, MD, 20817, US

(21) Appl. No.: 10/296,175

(22) PCT Filed: May 22, 2001
(86) PCT No.: PCT/EP01/05890

(30) Foreign Application Priority Data
May 22, 2000 (DE) 100 24 992.2

(51) Int. Cl. G01N 33/53

(52) U.S. Cl. 435/7.1

(57) ABSTRACT

A method for determining substrate and product concentration in liquid and/or gaseous media is disclosed. The analytes are removed by time-controlled diffusion of at least one analyte between the medium and a diffusion medium which is fed to the sampling regions through fluid conduits using at least one pump and semipermeable membranes. The diffusion medium is transported to at least one detector, while simultaneously new diffusion medium is fed from the sampling region and analyzed to determine the analyte concentration. The pump operates continuously and the diffusion medium is fed to the fluid conduits in alternation through a multivalve or multipath valve connected in series upstream from the sampling regions. The detector and the pump are additionally connected through a bypass conduit joined to the valve, and the detector is continuously fed diffusion medium through the fluid conduits and the bypass conduit.

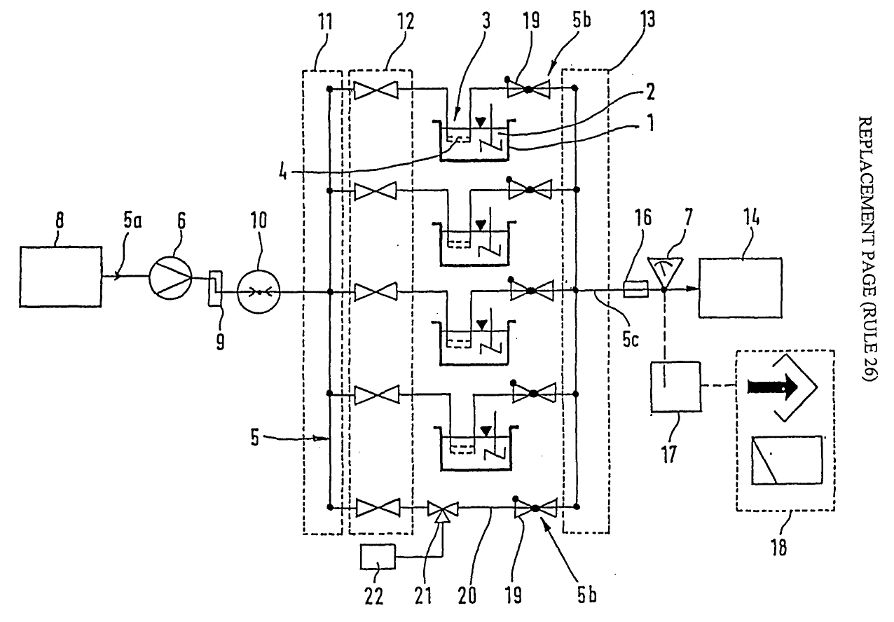

The present invention concerns a method for determining substrate and product concentrations in liquid and/or gaseous media in which several samples are taken of at least one substance to be analyzed-the analyte-in at least one sampling region by time-controlled diffusion of the at least one analyte between the respective medium and a diffusion medium which is fed to the sampling region by fluid conduit segments using at least one pump through semipermeable membranes. Subsequently the diffusion medium is transported from the sampling region to at least one detector to which a new diffusion medium is fed in at the same time, and is analyzed by this to determine the analyte concentration, whereby the pump operates continuously and the diffusion medium is fed in alternation to the fluid conduit segments through a multivalve or multipath valve arrangement connected in series upstream from the sampling regions. Furthermore the invention concerns a device for implementing the method.

In various regions of natural and engineering science, especially in biology and chemistry as well as in biological and chemical process and environmental engineering, it is necessary and desirable to measure the concentration of certain substances in a large number of reaction mixtures at the same time, whereby in particular a particular significance accrues to online analysis.

Known approaches in this connection consist in allocating a sensor arrangement with a complete metering pipe to one reaction container each whereby sensors are used which can be introduced directly in a fluid stream escaping from this. Here the precondition is that the sensor can come into contact with the medium in which the concentration of a substance is to be measured, that is, is not directly attacked by the medium. Furthermore the limiting conditions, for example, the pH value and temperature, permit direct application and a sufficiently exact measurement in the undiluted medium. Many sensors do not meet these technical preconditions. For example, with sensors with immobilized enzymes, the instability of the enzyme, above all at higher temperatures (steam sterilization), and the restricted measurement range prevent direct application. For these reasons, the detectors are arranged outside the reactor container in known online analysis apparatus. Sampling takes place here regularly in that volumes of medium are removed from reaction containers to be sampled and fed to an analysis device or detector through transport conduits. A frequent volume sampling is nonetheless possible only in containers in which the volume removed is very small in relation to the reaction volume. That means that with this sampling strategy, the frequencies and extent of sampling depend upon the reaction volume and are directly restricted by it.

For this reason, conducting sampling by time-controlled diffusion of the analyte from the medium to be sampled into an acceptor liquid through dialysis tubes is proposed in DE 197 29 492 A1. Moreover, the enrichment of the analyte into the diffusion medium and therewith the sampling is controlled through diffusion time. This procedure has the advantage that only molecules are removed from the medium, but no volume of medium. Sampling is thus limited only by the overall amount of substance and not by the reaction volume.

With the known methods, the transport of the acceptor fluid through the facility takes place using a pump. This is turned off after filling the dialysis tubes with fresh acceptor fluid so that analytes which are present in the reaction range in higher concentrations are accommodated by diffusion into the acceptor fluid through the walling of the dialysis tube. This method consequently requires an actuation of the pump which must be turned on and off, and in addition an actuation of values arranged in the sampling regions if need be. Moreover, it is not optimal that a continuous conveyance does not take place during starting times when starting the pump, or when stopping the pump, and correspondingly the turning on and turning off times cannot be used.

Alternatively, using a continuously operating pump and actuating the fluid conduit segments in alternation through a multipath valve is proposed in Talanta, Vol. 49 (1999), p. 403-414. In this way, a transport of the acceptor from a sampling region to the detector can take place in a simple manner since that fluid conduit segment which contains the sampling region is connected with the pump through corresponding actuation of the multipath or multivalve valve arrangement. Nonetheless, even here times can occur in which the pump must be shut off since no sampling section is being flowed through.

The object of the invention therefore is to refine a method of the type mentioned at the beginning such that analysis series can be effectively undertaken with low expenditure.

This objective is accomplished in that the detector and the pump are additionally connected through a bypass conduit which is in particular connected to the valve arrangement, and diffusion medium is continuously fed to the detector over the fluid conduit segments and the bypass conduit.

In accordance with the present invention, a bypass conduit is consequently provided through which diffusion medium is guided by the pump past the sampling region to the detector. This bypass can likewise be actuated through a multivalve or multipath valve arrangement and alternatively to the sampling regions. Diffusion medium, for example, can be conducted due to the presence of such a bypass conduit if at one time no sampling region is to be flowed through. In this way, the pump can operate continuously. Turning it off and on is no longer necessary. It is also possible to inject a standard medium into the diffusion medium in the region of the bypass and to transport this segment by connecting the bypass to the detector. By conducting this process repeatedly before and during the duration of the test, drift phenomena of the detector can be corrected.

Furthermore, the detector can be fed rinsing fluid through the bypass conduit.

An especially efficient sampling is attained if in parallel sampling regions in any given case the diffusion or sampling time of a region is at least the measuring time necessary for signal recording in the detector of all other parallel sampling regions together. The diffusion times are correspondingly adjusted to one another such that during sampling in one region, the measurements for the other sampling regions can be undertaken simultaneously one after the other and then the measurement of the sample can then also be directly joined to the diffusion. In this way, an especially high effectiveness and flexibility is attained.

It is provided in developing the invention that a pressure measurement takes place connected in series in front of the sampling regions in the conduit for the diffusion medium for recognition of a disturbance in a conduit segment. Underlying this is the consideration that, for example, when a leakage occurs in the conduits between the pump and the detector, a portion of the diffusion medium is not conducted through the detector, but rather into the defective conduit to the extent that conduit resistance is less in this direction than toward the detector. Sampling would thus not only be impaired in the defective region, but also in the overall system. Building in a pressure sensor makes possible here automatic disturbance recognition since the conduit pressure in connection with through flow of parallel regions moves in a value range characteristic for the device. If the pressure sinks outside the characteristic value range during flow through of one of the parallel conduit regions, a disturbance is present, namely in the event of an excessively low pressure, a leak, and in the event of an excessively high pressure, a stopping up, and the defective conduit region can be uncoupled.

In addition, a check valve can be connected after the sampling regions in series, or alternatively an additional multipath or multivalve arrangement can be provided which prevents a diffusion medium from flowing back from a sampling region into another sampling region.

Several detectors can be provided connected in series in an inherently familiar manner for simultaneous analysis of different analytes. Since according to experience, the detectors can also fail or sharply drift, it can also be appropriate to provide several detectors for the same analytes in parallel regions which can be turned on as a replacement in the event a detector fails. Electively, however, various detectors can also be connected parallel through a multipath or multivalve valve arrangement, owing to which the possibility is opened of determining different analytes at various points in time. Such an interconnection, for example, is appropriate with detectors that mutually influence one another in their measuring processes.

In accordance with a further aspect of the invention, the device contains a sample reparation module connected in series in front of the detector which either absorbs isturbing components from the diffusion medium (for example, activated charcoal) or transforms them reactively into a non-disturbing chemical form. Alternatively or additionally, there are also detectors which require a sample preparation module so that the analyte is transformed into a detectable form (for example, enzyme or dye reactions and the photometric measuring method).

In a preferred manner, a diffusion medium is used which is basically free from the analytes to be detected so that the concentration gradient is high over the semipermeable membrane from the medium to be sampled to the diffusion medium. In cases in which the analyte concentration in the medium to be sample falls below the detection limit of a detector, it can, however, also be appropriate to use a diffusion medium which contains a known concentration of the analyte or analytes which lies above the low concentration in the medium. Then a diffusion of the analyte into the medium takes place in the region of the sampling region and the loss in concentration over diffusion time is measured in the diffusion medium and used for determining the analyte concentration in the medium to be sampled.

The diffusion medium can be eliminated before undertaking the analysis. Alternatively, it is also possible to collect the samples in an automatic fraction collector for a subsequent off-line analysis.

The components of a sample are quantified by being fed to appropriate detectors. Since is a matter of a relative measuring method in measuring the diffusively obtained sample segments, the measurement signals from unknown concentrations can only be ascertained in comparison with a standard mixture sampled through diffusion under operating conditions. Furnishing a standard solution into which a further semipermeable membrane is dipped separately from the other sampling sites, as this is known on the basis of DE 197 29 492 A1, does not suffice for such a calibration if the semipermeable membranes selected do not have exactly identical properties, such as, for example, the same length, surface and wall thickness. Empirically, such tubes are not to be manufactured so exactly in relation to these features with the consequence that a signal measured in the detector of a sample diffusively enriched in this manner cannot be relied upon for calibration. In accordance with the present invention, it is therefore provided that, for calibration, the semipermeable membranes are dipped in media of known analyte concentration, and in each case measurement data sets are compiled on the basis of which the measurement results furnished by the detector are evaluated for determining the analyte concentration.

Especially with sterile technique requirements, standard concentrations can also be directly deposited in the reaction containers. This prevents frequent medium changes and expensive sterilization measures in the containers. For this, known concentrations of at least one analyte are deposited into the preferably analyte-free medium through the addition of correspondingly calculated volumes of a concentrated standard mixture of the analyte. Then the reaction containers are sampled in the manner described above and corresponding measurement data sets are compiled. A renewed additional dosing of standard mixture into the reaction medium and subsequent measurement can be repeated until the highest concentration of analytes desired by the user is reached. In this way, the measurement range of analyte to be expected can be covered during the experiment.

The detector used can internally already be so adjusted or precalibrated that it directly determines the analyte concentrations in the samples passed through which are obtained through diffusion in the sampling regions. That is, the device supplies without further recalculation the analyte concentration present in the diffusion medium. On the basis of these analyte concentrations and the calibration method previously described, inferences can be made on the basis of these concentrations in the diffusion medium about the concentrations in the sampled medium with corresponding calculation models.

Alternatively, the detector can provide a temporal concentration distribution or a temporal distribution of a signal proportional to the concentration, whereby then an inference can be made as to the analyte concentration in the sampled medium though the calibration and a corresponding evaluation of the detector signals. Here the maximal rise of the front face of the detector signal or the elevated baseline following flow through of the peak maximum which results from the diffusion of the analyte at constant through flow (volume flow) into the diffusion medium are adduced for evaluation.

In constructing this embodiment, it can in particular be provided that a change in the ratio of the signal maximum to the base line in the output of the detector is ascertained, and on the basis of it, a change in the diffusion properties of the semipermeable membranes is inferred and a corresponding correction factor is ascertained, and taken into account in further processing. In this way, a possible drift owing to change in diffusion properties, for example due to blockages or coatings (fouling) can be allowed for through data evaluation.

Alternatively or additionally, it is possible to ascertain two signals at different flow rates of the diffusion medium and/or different diffusion times with resting medium in close temporal sequence and to compare them with one another with respect to their characteristic properties in order to recognize on this basis a possible drift due to fouling which then can be appropriately taken into consideration in connection with data evaluation. In addition, in this case, a temporal change in analyte concentration known from several measurements and/or a dynamic model can be taken into consideration.

With respect to further advantageous refinements of the present invention, reference is made to the dependent claims as well as to the following description of an embodiment of a device with which the process of the invention can be conducted on the basis of the drawing.

In the drawing, the sole FIGURE shows in schematic representation a device for determining substrate and production concentrations in liquid and/or gaseous media 2. The device has a large number of reaction containers 1 in which in any given case a gaseous or liquid medium 2 to be analyzed is contained. With the reaction containers 1, it can, for example, be a matter of vibration cylinders which are kept constantly in motion. Through analysis, the concentrations of substances, of extracts or of reaction products, hereinafter called analytes, are measured inside the medium.

At least one sample module 3 is set in each reaction container 1 which has a semipermeable membrane 4 which is here constructed in the form of a dialysis tube and is dipped completely into the medium 2 contained in the reaction container 1. The dialysis tubes 4 are arranged in the manner of a parallel connection and connected inlet-side through fluid conduits 5 with a pump 6 and outlet-side with a detector 7. The pump 6 is connected with a storage container 8 for accommodating a diffusion medium suited for a diffusion sampling which can be gaseous or liquid as a function of the physical condition of the medium 2 to be sampled. A bubble trap 9 is provided arranged after the pump 6 which serves to remove bubbles from liquid diffusion medium. Moreover, a pressure sensor 10 is provided which measures conduit pressure.

The fluid conduit segment Sa coming from the pump 3 opens into a medium distributor 11 to which the parallel fluid conduit segments 5b are connected outlet side with sampling module 3, and a multivalve arrangement 12 is provided between the media distributor 11 and the sampling module 3 through which the parallel fluid conduit segments 5b are opened in each case for a flow through of diffusion medium or and can be closed for preventing such a through flow.

On the outlet side, the sampling modules 3 open through the parallel fluid conduit segments 5b into a media collection module 13 which has on its outlet side a discharge 5c which leads to a detector 7 and an outflow lying behind it into a suitable waste reservoir 14 or into another type of drain for the diffusion medium. In the outflow 5c, a sampling preparation module 16 is provided before the detector viewed in the direction of flow which absorbs disturbing components from the diffusion medium or reactively transforms them into a non-disturbing chemical form. Alternatively or additionally the sample preparation module 16 can also serve to transform the analyte into a form which can be recorded by the detector 7.

The signal output of the detector 7 is connected with a computer 18 through a measurement amplifier 17 which evaluates the measurement signals originating from the detector 7 and moreover also controls the valves of the multivalve arrangement 12 as well as the rate of conveyance of the pump 6.

Moreover stop valves 19 are provided in the fluid conduit segments 5b between the sampling modules 3 and the media collecting module 13 which in the event of a leakage in a fluid conduit segment 5b are supposed to prevent the diffusion medium which comes from a sampling module 3 from flowing into the defective conduit segment 5b instead of toward the detector.

In addition to the parallel sampling regions with the sampling modules 3 provided therein, a bypass conduit 20 is connected through a further valve of the multivalve arrangement 12 through which diffusion medium can be guided from the pump 6 past the sampling regions 5b to the detector 7. In this way, for example, the baseline of the detector 7 can be ascertained when fresh diffusion medium flows through or the detector 7 is rinsed with a rinsing agent through, for example, a pump connected only to the bypass 20. In addition, in the bypass, the possibility of introducing a sample segment of a standard mixture which is contained in a storage container 22 into the flow of the diffusion medium is provided through a three/two way valve 21 or another type of injection valve.

Parallel sampling with the device of the invention takes place as described below:

First of all, a suitable diffusion medium is pumped into the facility with the aid of the pump 6 until the fluid conduits 5 as well as the dialysis tubes 4 are completely filled with the diffusion medium. Proceeding from this setting, a sampling takes place in each case in the sampling modules 3 since with a continuously operating pump 6, the valve of the multivalve arrangement 12 connected in series in front of the corresponding fluid conduit region 5b is closed so that the diffusion medium rests in the sampling module 3 of this fluid conduit region 5b. This condition is maintained during a specified duration so that by diffusion, an adaptation of the concentrations of the analyte in the medium to be sampled, which is contained in reaction container 1 and the diffusion medium, takes place. If one proceeds from the assumption that the analyte concentration in the medium to be sampled is higher than in the diffusion medium, an amount of analyte characteristic for the concentration of the analyte in the medium accumulates in the diffusion medium within the specified time period. If the analyte concentration is higher in the diffusion medium, an analyte enrichment takes place in this in a reversed manner though the diffusion taking place. Moreover, it is advantageously assured in contrast to filtration that the volume of the medium contained in the reaction container 1 basically remains unchanged.

After lapse of the specified period of time, the valve of this fluid conduit region 5b is opened once again so that the diffusion medium contained in the dialysis tube 4 enriched or depleted with analyte is transported to the detector 7, and at the same time new diffusion medium flows back into the fluid conduit region 5b. The sample segment is analyzed when it flows through the detector 7, whereby the detector 7 emits measurement signals to the computer which correspond to the respective concentrations of analyte in the allocated reaction containers 1. In what way the evaluation takes place is yet to be explained below.

A sampling can be undertaken in the previously described manner in all sampling modules 3 by diffusion between the medium 2 contained in the respective reaction container 1 and the diffusion medium and an analysis can subsequently be undertaken since the segment of the diffusion medium which is subjected to diffusion in the sampling module 3 is transported to the detector 7 and analyzed by this when it flows through. The analysis of the sample segments received in the individual sampling regions takes place in alternation one after the other, that is, time-shifted. In order to be able to conduct measurements nonstop, that is, continuously with the least loss times possible, the measurement times are in each case determined such that the measurement or transport time in one of the parallel fluid conduit segments 5b is equal to the sum of the diffusion times of the other parallel regions, or, conversely, the diffusion and sampling time of one region is at least the measuring time necessary for signal recording in the detector for all other parallel sampling regions together. In other words, the diffusion times on the one hand and the measurement times on the other are so harmonized with one another that, with the exception of some connection-conditioned delays, one of the parallel fluid conduit segments 5b is flowed through and correspondingly, the segment of diffusion medium which was previously subjected to diffusion is analyzed.

The detector can internally already be adjusted or pre-calibrated such that it directly determines the analyte concentrations in the samples passed through out of the sampling modules 3. That is, it provides the analyte concentration present in the diffusion medium without further recalculation. On the basis of these analyte concentrations, it is possible to infer back to the analyte concentration contained in the sampled medium by calculation on the basis of measurement series which were obtained in the framework of a previously conducted calibration.

Alternatively, the detector can provide a temporal distribution of the concentration of sample flowing through (dwelling time curve) or a temporal distribution of a signal proportional to the concentration. On the basis of measured value series which were obtained in a previously conducted calibration process, an inference as to the analyte concentration in the sampled medium can be ascertained whereby various properties can be adduced for the evaluation, such as, for example, the peak maximum, an increase of the front face, the area under the curve, the base line in the downflow of the curve, etc. Since such analysis methods are basically known, they will not be gone into in detail here. Only for the sake of completeness is reference made in this regard to the content of the disclosure of DE 197 29 492 A1.

As already mentioned, the analyte concentration in the diffusion medium is measured and an inference is subsequently made on the unknown analyte concentration in the sampled medium 2. Since here it is a mater of a relative measurement process, a precalibration must take place in which the analyte concentration in the diffusion medium is placed in relation with the analyte concentration in the medium to be sampled 2.

Before conducting the series of tests, each sampling module 3 is dipped in at least one medium with known analyte concentration for this. With the same diffusion times and other adjustments of a device as in the planned experiment, the measurement is now conducted in each connected sampling module 3. In this way, a set of measurement data for each analyte is allocated to each sampling module. The connection so obtained between the concentration in the reaction container to be sampled and the detector response during transport of the sample obtained by diffusion through the detector is used for evaluating the computer signals obtained online in the experiment.

The precalibration can also be undertaken directly in the reaction container 1, since there a specific volume of a concentrated standard analyte mixture of known construction (which is preferably is mixed with the medium to be sampled in order to prevent the dilution of other components of the medium) is dosed in. Thus a concentration known through the dosing in arises. Then the reaction containers 1 are sampled in the manner described above and the measurement data are recorded. A renewed dosing in of the standard analyte mixture into the medium to be sampled and subsequent measurement can be repeated so often until the highest concentration of the analyte desired by the user is reached. Thus the expected measurement range of the analyte can be covered during the experiment.

In addition to the precalibration, an intermediate calibration can take place through the bypass 20 while the experiment is running. Alternatively, a sampling module arranged in a bypass or in a further parallel region can be dipped in a standard mixture during the experiment and can be sampled at regular intervals for recalibration with the same diffusion time.

If a leak occurs during the time of the experiment in the fluid conduits 5 or the sampling modules 3, the diffusion medium could be passed through the medium collection module 13 from other sampling regions 5b not through the detector 7, but rather into the defective region insofar as the conduction resistance is less in this direction than in detector 7. Sampling would thus be impaired not only in the defective fluid conduit region 5b, but also in the entire system. The stop valves 19 or a multivalve arrangement to be used as an option arranged in the device prevent this.

With a liquid diffusion medium, the gauge in the medium to be sampled will rise in the event of a leak inside reaction container 1, and the medium is necessarily contaminated in this case. With a gaseous diffusion medium, the pressure can rise in a closed container 1.

With leaks outside the reaction container 1, recognition of the leak is obvious with liquids, but not with gases, for example. Disturbances in the conduit can be recognized by incorporating a pressure sensor 10. This is based upon the consideration that the conductance pressure in the parallel fluid conduit regions 5b moves in a value range characteristic for the device when parallel regions and the bypass 20 are flowed through. If the pressure lies outside these ranges when fluid flows through a conduit region 5b, there exists a disturbance and the defective region 5b can then be uncoupled, that is, no longer subject to through current. Concretely, when pressure is too low, there is a leak, and when it is too high, there is a stoppage.

If the diffusion properties of the dialysis tubes 4 or another semipermeable membrane worsen during the experiment, for example by coating of its surface with components from the medium to be sampled (fouling), then the concentration equalization will be less than without this coating with a specified sampling time (diffusion time) in the dialysis tubes 4. This error is recognizable by evaluating the detector signals and can be incorporated into the concentration calculation with the original calibration values as an online corrector. The signal which is recorded when a detector 7 is subjected to through blow is, for example, a peak which does not return to the base line level in the event that pure diffusion medium flows through. The signal approaches a level which arises when diffusion medium flows through the sampling module 3 through diffusion in connection with a through current (effect of a contact time- and therewith disturbance-dependent diffusion). In this way, the enrichment in diffusion medium is known at two different diffusion times. From the comparison of these two values and the change in their ratio to each other, the changes in diffusion properties, that is fouling, can be taken into consideration in the measurements and be corrected by calculation.

A single detector 7 is used in the device represented in the drawing. Since such a detector 7 can fail or drift sharply, optimally several detectors can be provided for the same analyte which can be electively turned on or turned off, for example in the event of the failure of a detector 7. Electively different detectors can also be connected parallel, for example through multipath or multivalve arrangements so that various analytes can be analyzed at various points in time. Such an interconnection is, for example, appropriate in detectors which mutually influence one another in their measuring process.

In sum, the previously described device operates in a very efficient manner since a measurement can take place practically continuously in the detector 7 provided, whereby when the pump 6 operates continuously, the individual parallel sampling regions 5b are opened for a transport of diffusion medium or closed during the diffusion time simply through actuation of the multivalve arrangement 12. Of course, it is also possible to use several pumps for the diffusion medium.

1. Method for determine of substrate and product concentration in liquid and/or gaseous media in which several samples of at least one substance to be analyzed-the analyte-are removed in at least one sampling region (3) by time-controlled diffusion of the at least one analyte between the respective medium and a diffusion medium which is fed to the sampling regions (3) through fluid conduit segments (5a, 5b) using at least one pump (6) by semipermeable membranes (2) and subsequently the diffusion medium is transported to at least one detector (7) while simultaneously new diffusion medium is being fed from the sampling region (3) and is analyzed by this to determine the analyte concentration, whereby the at least one pump (6) operates continuously and the diffusion medium is fed to the fluid conduit segments (5b) in alternation through a multivalve or multipath valve arrangement (12) connected in series upstream from the sampling regions (3), characterized in that the detector (7) and the pump (6) are additionally connected through a bypass conduit (20) which is in particular joined to the valve arrangement (12), and the detector is continuously fed diffusion medium through the fluid conduit segments (5b) and the bypass conduit (20).

2. Process according to claim 1, characterized in that in the parallel sampling regions (3), the diffusion and sampling time of one region in any given case are at least the measuring time necessary for signal recording in the detector of all other parallel sampling regions together.

3. Process according to claim 1 or 2, characterized in that in the region of the bypass conduit (20), a standard medium is injected into the diffusion medium and this segment is transported by connecting the bypass conduit (20) to the detector (7) in order to correct drift phenomena of the detector (intermediate calibration).

4. Process according to claims 1 to 3, characterized in that rinsing fluid is fed to the detector through the bypass conduit (20).

5. Process according to one of the preceding claims, characterized in that a pressure measuring unit is connected in series upstream from the sampling regions (3) in the fluid conduit (5a) for recognition of a disturbance in a conduit segment.

6. Process according to one of the preceding claims, characterized in that air or gas bubbles are removed from fluid diffusion medium before reaching the sampling regions (3) using a bubble trap (9).

7. Process according to one of the preceding claims, characterized in that the multipath or multivalve arrangement (12) is controlled by a computer (18).

8. Process according to one of the preceding claims, characterized in that several parallel connected detectors are provided and diffusion medium coming from the sampling regions is fed to one of the detectors through a multipath or multivalve arrangement.

9. Process according to one of the preceding claims, characterized in that in a sample preparation module (16) connected upstream in series from the detector (7), at least one substance which can disturb the detector used is absorbed or reactively transformed into a non-disturbing chemical form.

10. Process according to one of the preceding claims, characterized in that in a sample preparation module (16), the analyte is reactively transformed into a form measurable by the detector (7).

11. Process according to one of the preceding claims, characterized in that a diffusion medium is used which is basically free from analytes to be detected.

12. Process according to one of the preceding claims, characterized in that a diffusion medium is used which contains a known concentration of at least one analyte which lies above the concentration in the medium to be sampled so that a diffusion of the analyte from the diffusion medium into the medium to be sampled takes place in the region of the sampling region.

13. Process according to one of the preceding claims, characterized in that the samples obtained from parallel sampling regions through diffusion are gathered with an automatic fraction collector in the output of the sampling region or of the detector for a subsequent off-line analysis.

14. Process according to one of the preceding claims, characterized in that, for calibration, the semipermeable membranes are dipped in media of known analyte concentration and measurement data sets are compiled on the basis of which the measured results supplied by the detector are evaluated for determining analyte concentration.

15. Process according to one of claims 1 to 14, characterized in that the semipermeable membranes (2) are dipped for calibration in at least one reaction container with the medium to be used in the experiment, and in that known concentrations of at least one analyte is set through the addition of correspondingly calculated volumes of a concentrated standard mixture of at least one analyte and measured data sets are compiled for the various concentrations on the basis of which the measurement results supplied by the detector are evaluated for determining the analyte concentration.

16. Process according to claim 14 or 15, characterized in that the end concentration of the at least one analyte at the same time represents the desired start concentration in the experiment mixture.

17. Process according to one of claims 13 to 15, characterized in that the detector (7) issues a value for the concentration of the analyte in the diffusion medium and an inference is made by calculation about the concentration in the medium at past diffusion times by comparing this measured value with the measured values which were ascertained by the calibration method according to claims 18 to 20 with a known analyte concentration.

18. Process according to one of the preceding claims, characterized in that the detector supplies a temporal concentration distribution or a temporal distribution of a signal proportional to the concentration.

19. Process according to claim 18, characterized in that an inference is made as to the analyte concentration in the sampled medium through calibration in accordance with claims 13 to 15 and a corresponding evaluation of the detector signals, whereby the maximum rise of the front face of the detector signal, the signal maximum, the surface under the signal curve or the elevated baseline following through flow of the peak maximum which results from the diffusion of the analyte into the diffusion medium are adduced for the evaluation.

20. Process according to claim 19, characterized in that several properties of the detector signal distribution are used for the evaluation simultaneously, or ratios of these values to one another are used.

21. Process according to claim 19 or 20, characterized in that a change in the ratio of the signal maximum to the baseline is ascertained in the output of the detector signal and on the basis of this an inference is made on a change in the diffusion properties of the semipermeable membrane and a correction factor is ascertained.

22. Process according to one of the preceding claims, characterized in that two signals at different rates of flow of the diffusion medium and/or different diffusion times in the resting medium are ascertained in close temporal sequence and compared with one another with regard to their characteristic properties in order to recognize and to correct a possible drift through a change in diffusion properties.

23. Process according to claim 22, characterized in that in addition, a change over time in analyte concentration known on the basis of several measurements and/or a dynamic model is considered.

24. Device for implementing the method according to one of claims 1 to 23, with several reaction containers (1), which are arranged in the manner of a parallel connection and are connected inlet-side with a pump (6) and outlet-side with a detector (7) through fluid conduits (5a, 5b, 5c), whereby a multivalve or multipath valve arrangement (12) is provided in the fluid conduit segments (5b) between the pump (6) and the semipermeable membrane (2) through which in any given case one of the parallel fluid conduit segments (5b) between the pump (6) and the detector (7) can be opened in order to subject in alternation the parallel fluid conduit segments (5b) to the detector (7) at all time to a diffusion medium flowing through, characterized in that a bypass conduit (20) is provided between the pump (6) and the detector (7), through which diffusion medium can be guided past the sampling regions (3) to the detector (7).

25. Device according to claim 24, characterized in that the bypass conduit (20) is connected to the multivalve or multipath valve arrangement (12).

26. Device according to claim 24 or 25, characterized in that at least one injection valve is connected to the bypass (20).

27. Device according to one of claims 24 to 26, characterized in that a pressure sensor is arranged in the fluid conduit (Sa) downstream from the semipermeable membranes (2).

28. Device according to one of claims 24 to 27, characterized in that a bubble trap (9) is arranged in the fluid conduit segment (Sa) connected upstream from the multivalve or multipath valve arrangement (12).

29. Device according to one of claims 24 to 28, characterized in that a sample preparation module (16) is provided in the fluid conduit segment (5c) between the detector (7) and the semipermeable membranes (2) to absorb certain substances or reactively transform them.

30. Device according to one of claims 24 to 29, characterized in that a sample preparation module (16) is provided in the fluid conduit segment (5c) between the semipermeable membranes (2) and the detector (7) to transform at least one analyte into a form measurable by the detector (7).

31. Device according to one of claims 24 to 30, characterized in that in any given case a stop valve (19) is provided arranged in the parallel fluid conduit segments (5b) after the semipermeable membranes (2).

32. Device according to one of claims 24 to 31, characterized in that the membranes (2) open outlet-side into a medium collecting module (12) which connects at least two of the parallel fluid conduit segments (5b) with a discharge tube (5c) which leads to at least one detector (7).
